(19) United States
(12) Patent Application Publication
(10) Pub. No.: US 2026/0074053 A1
(43) Pub. Date: Mar. 12, 2026

(54) METHODS AND SYSTEMS FOR STRENGTH-BASED COPING SKILS TRAINING FOR PERSONS ON THE AUTISM SPECTRUM

(71) Applicant: Kessler Foundation Inc., East Hanover, NJ (US)

(72) Inventor: Helen Genova, Fanwood, NY (US)

(21) Appl. No.: 19/323,080

(22) Filed: Sep. 9, 2025

Related U.S. Application Data
(60) Provisional application No. 63/692,891, filed on Sep. 10, 2024.

(51) Int. Cl. G16H 20/70

(52) U.S. Cl. CPC G16H 20/70

(57) ABSTRACT

Exemplary method for strength-based coping training includes (a) prompting user, by a software application operable on a computing device, to identify at least one of their top character strengths from a preset list of character strengths, the preset list of character strengths being identified in a character strengths database; (b) receiving from the user in response, a selection of at least one of their top character strengths from the preset list of character strengths; (c) processing the received selection of the user's top character strengths to identify different coping strategies from a coping strategy database that the user may wish to consider, wherein identification of the different coping strategies being based on the received user's top character strengths selection; and (d) the software application providing to the computing device information for presenting, or making accessible, to the user the identified different coping strategies that the user may wish to consider.

### CROSS-REFERENCE TO RELATED APPLICATION

This application, which claims priority from U.S. provisional application no. 63/692,891 filed on Sep. 10, 2024, which is hereby incorporated by reference herein in its entirety.

### FIELD OF THE INVENTION

This patent specification generally relates to the field of coping-skills training, and more specifically, to computer-implemented methods for coping-skills training, especially for trainees on the autism spectrum, based on identification and use of a trainee's character strengths.

All publications and other references cited in this application are incorporated herein by reference in their entirety for all purposes and to the same extent as if each individual publication or other reference was specifically and individually indicated to be incorporated by reference in its entirety for all purposes. Citation of a reference herein shall not be construed as an admission that such is prior art to the present invention.

### BACKGROUND

An estimated 1 in 49 youth are diagnosed with autism spectrum disorder, a developmental condition characterized by challenges in social and emotional functioning. Transition age youth on the autism spectrum, ages 12-21 (“TAY-AS”) can experience difficult in regulating emotions and coping when stressed and overwhelmed 2-9. While traditional mental health approaches, like Cognitive Behavioral Therapy (CBT), provide coping strategies, it is unclear whether these approaches have been and are particularly beneficial for TAY-AS. Further, coping strategies taught during traditional “talk therapy” can be inaccessible to TAY-AS when highly stressed or feeling overwhelmed.

Strength-based coping has been shown to have clinical benefit across multiple populations including youth ages 18 to 20. See for example, a recent study reported in Toback, R. L., Graham-Bermann, S.A. & Patel, P.D., Outcomes of a character strengths-based intervention on self-esteem and self-efficacy of psychiatrically hospitalized youths, Psychiatr. Serv. 67, 574-577 (2016), that notes the benefit of a strength-based coping intervention in youth hospitalized for psychiatric reasons. In this study, the youth in the treatment group wrote strengths-based coping strategies on index cards to carry and use during times of stress. Following the treatment, youth reported improved self-efficacy and self-esteem, as well increased use of their strengths as coping mechanisms.

A need exists for improved and more accessible methods for coping training, especially for trainees on the autism spectrum.

### BRIEF SUMMARY OF THE INVENTION

The embodiments of the invention disclosed herein rely on an easily administered strength-based approach by having users (a) identify their strengths within a computer application executed by a desktop computer and/or mobile device as well as remote computer via the desktop computer, or mobile device, (b) choose strength-based coping strategies and then (c) use the strategies during times of stress. The embodiment of the invention disclosed herein advantageously eliminate the need for physical cards, which may be lost or destroyed as the app will be within hands-reach of the user.

The inventor believes that strength-based coping can be taught through a positive psychology interventional framework in which an individual 1) identifies their personal strengths and 2) purposefully uses them to cope with the stressors of life.

According to one embodiment of the invention, a method for strength-based coping training includes by a computing device executing a software application, (a) prompting a user to select at least one of their top character strengths from a preset list of character strengths, wherein the preset list of character strengths being identified in a character strengths database accessible by the software application; (b) receiving from the user in response to the prompting, a selection of at least one of their top character strengths from the preset list of character strengths; (c) processing the received selection of the user's top character strengths to identify different coping strategies that the user may wish to consider from a coping strategy database associated with the software application, wherein identification of the different coping strategies being based on the received user's top character strengths selection; and (d) providing to the computing device information for at least one of presenting or making access to the user the identified different coping strategies that the user may wish to consider.

The method continues by (e) the computing device prompting the user to select a predetermined number of the identified coping strategies; (f) receiving from the user in response to the prompting by the software application, (i) the user's selection of the identified coping strategies for each of the selected user's top character strengths, and (ii) information indicative of personalized goals with regard to use of the selected coping strategies; storing in a user database the user's selected coping strategies for each of the selected user's top character strengths and the information indicative of personalized goals with regard to use of the selected user's top character strengths; (g) storing in a user database the user's selected coping strategies for each of the selected user's top character strengths and the information indicative of personalized goals with regard to use of the selected user's top character strengths; (h) receiving from the user, a request to access at least one of the stored selected coping strategies; (i) prompting the user to provide information of how the user feels after use of at least one of the coping strategies; and (j) processing the received information of how the user feels after use of at least one of the coping strategies, and prompting the user to different one of the at least one of the coping strategies, when the received information of how the user feels indicative of little or no improvement in feelings.

According to an alternative embodiment, the preset list of character strengths includes twenty-four character strengths.

According to an another embodiment, the step of prompting by a software application in step a. further comprises prompting the user to select three to five of their top character strengths.

According to a further embodiment of the invention, the method is specifically adapted for coping training of users on the autism spectrum.

### BRIEF DESCRIPTION OF THE DRAWINGS

Certain embodiments of the present invention are illustrated as an example and are not limited by the figures of the accompanying drawings, in which like references may indicate similar elements and in which:

FIG. 1 depicts an illustrative example of some of the components and computer server-implemented methods that may be found in a system for practicing various embodiments described herein.

FIG. 2 illustrates a block diagram showing an example of software components maintained by a computer server as described with regard to various embodiments herein.

FIG. 3 illustrates a block diagram of an exemplary computing device that may be used by with the computer server of FIG. 2.

FIG. 4 illustrates a flow diagram of a representative example of a computer-implemented method for character strength-based coping training according to various embodiments described herein

FIG. 5 is an exemplary chart of twenty-four different character strengths useable with the method depicted in FIG. 4.

FIG. 6A is a representative flow diagram of an exemplary sequence of steps for onboarding users and after onboarding, execution of a representative version of the exemplary method depicted in FIG. 4.

FIG. 6B depicts an exemplary sequence of display screens useable on a mobile user device for performing the exemplary depicted in FIG. 4.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It should be understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

### DEFINITIONS

As used herein, “character strengths” means generally a person's positive traits and qualities reflected in their thoughts, feelings, personality, and actions, and can include capacities for thinking, feeling, and behaving, which can be expressed in degrees.

As used herein, the term “computer” refers to a machine, apparatus, or device that is capable of accepting and performing logic operations from software code. The term “application”, “software,” “software code,” or “computer software” refers to any set of instructions operable to cause a computer to perform an operation. Software code may be operated on by a “rules engine” or processor. Thus, the methods and systems of the present invention may be performed by a computer or computing device having a processor based on instructions received by computer applications and software.

The term “computing device” or “user device” as used herein is a type of computer or computing device accessible to the user that comprises circuitry and configured to generally perform functions such as recording audio, photos, and videos; displaying or reproducing audio, photos, and videos; storing, retrieving, or manipulation of electronic data; providing electrical communications and network connectivity; or any other similar function. In some embodiments, a computing device is a smartphone or computer configured to receive and transmit data to a server or other electronic device which may be operated locally or in the cloud. Non-limiting examples of computing devices include: personal computers (PCs), workstations, laptops, tablet PCs including the iPad, mobile phones including iOS phones made by Apple Inc., and Android OS phones, such as Samsung Galaxy phones, or any electronic device capable of running computer software and displaying information to a user, memory cards, other memory storage devices, digital cameras, external battery packs, external charging devices, and the like. Certain types of computing devices are portable and can easily carried by a person may sometimes be referred to as a “portable electronic device” or “portable device.” Some non-limiting examples of portable devices include cell phones, smartphones, tablet computers, and laptop computers.

The term “computer readable medium” as used herein refers to any medium that participates in providing instructions to the processor for execution. A computer readable medium may take many forms, including but not limited to, non-volatile media, volatile media, and transmission media. Non-volatile media includes, for example, solid-state drives, optical, magnetic disks, and magneto-optical disks, such as the hard disk or the removable media drive. Volatile media includes dynamic memory, such as the main memory. Transmission media includes coaxial cables, copper wire and fiber optics, including the wires that make up the bus. Transmission media may also take the form of acoustic or light waves, such as those generated during radio wave and infrared data communications.

As used herein the term “data network” or “network” shall mean an infrastructure capable of connecting two or more computers such as computing devices either using wires or wirelessly allowing them to transmit and receive data. Non-limiting examples of data networks may include the internet or wireless networks or (i.e. a “wireless network”) which may include Wi-Fi and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, or a voice-over-IP (VoIP) network.

As used herein, the term “database” shall generally mean a digital collection of data or information. The present invention uses novel methods and processes to store, link, and modify information such digital images and videos and user profile information. For the purposes of the present disclosure, a database may be stored on a remote server and accessed by a computing device through the internet (i.e., the database is in the cloud) or alternatively in some embodiments the database may be stored on the computing device or remote computer itself (i.e., local storage). A “data store” as used herein may contain or comprise a database (i.e. information and data from a database may be recorded into a medium on a data store).

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

New computer-implemented methods for character strength-based coping skills training are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

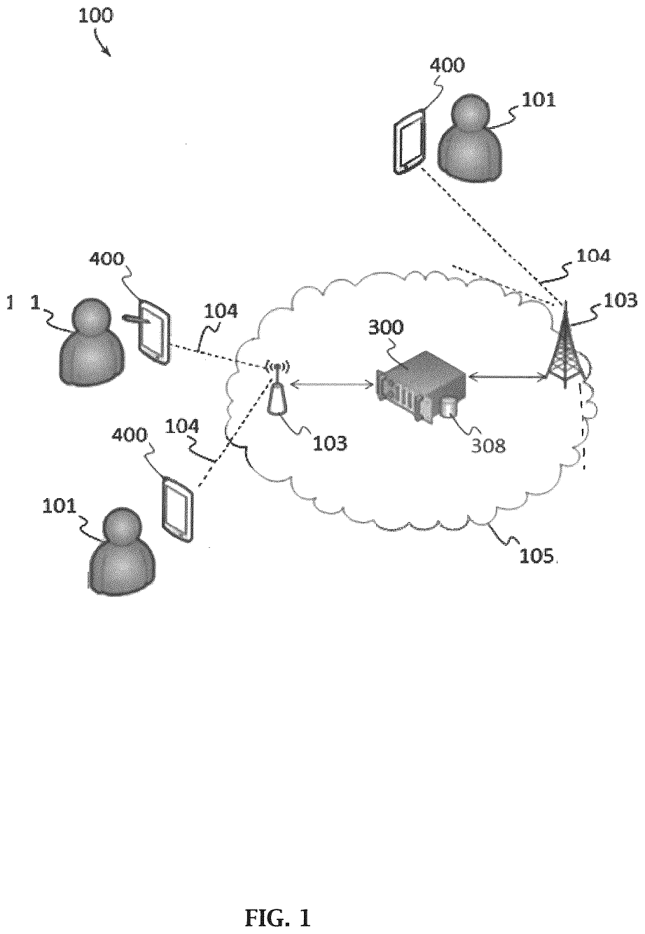

The present invention will now be described by example and through referencing the appended figures representing preferred and alternative embodiments. As perhaps best shown by FIG. 1, an illustrative example of a system 100 for implementing and use of methods for character strength-based coping skills training according to various embodiments described herein is presented. The system 100 is configured to facilitate the transfer of data and information between one or more access points 103, computing devices 400 of trainees, and servers 300 over a data network 105. Each computing device 400 may send data to and receive data from the data network 105 through a network connection 104 with an access point 103. Data stores 308 accessible by the server 300 may contain one or more databases. The data may comprise any information pertinent to one or more users, i.e., coping-skills trainees (the “trainees”) 101 that is input into the system 100 including information regarding the character strengths, and separately information regarding different occupations, and/or describing such users or trainees 101, information requested by trainees 101, information supplied by such trainees 101, and any other information which a trainee 101 may be provided in connection with the method for character strength-based coping skills training.

In this illustrative example, the system 100 comprises at least one computing device 400 configured to be operated by respective trainees 101. Computing devices 400 can be mobile devices, such as laptops, tablet computers, personal digital assistants, smart phones, and the like, that are equipped with a wireless network interface capable of sending data to one or more servers 300 with access to one or more data stores 308 over a network 105 such as a wireless local area network (WLAN). Additionally, computing devices 400 can be fixed devices, such as desktops, workstations, and the like, that are equipped with a wireless or wired network interface capable of sending data to one or more network-accessible computer servers 300 with access to one or more data stores 308 over a wireless or wired local area network 105. The present invention may be implemented on at least computing device 400, and/or server 300 programmed to perform one or more of the steps described herein. In some embodiments, more than one computing device 400, and/or server 300 may be used, with each being programmed to carry out one or more steps of a method or process described herein.

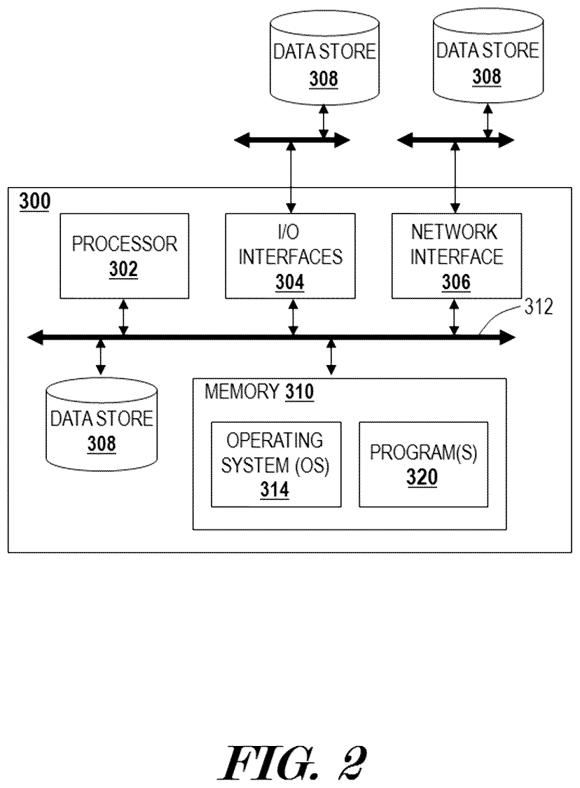

Referring now to FIG. 2, in an exemplary embodiment, a block diagram illustrates a computer server 300 of which one or more may be used in the system 100. The server 300 may be a digital computer that, in terms of hardware architecture, generally includes a processor 302, input/output (I/O) interfaces 304, a network interface 306, a data store 308, and memory 310. It should be appreciated by those of ordinary skill in the art that FIG. 2 depicts the computer server 300 in an oversimplified manner, and a practical embodiment may include additional components and suitably configured processing logic to support known or conventional operating features that are not described in detail herein. The components (302, 304, 306, 308, and 310) are communicatively coupled via a local interface 312. The local interface 312 may be, for example but not limited to, one or more buses or other wired or wireless connections, as is known in the art. The local interface 312 may have additional elements, which are omitted for simplicity, such as controllers, buffers (caches), drivers, repeaters, and receivers, among many others, to enable communications. Further, the local interface 312 may include address, control, and/or data connections to enable appropriate communications among the aforementioned components.

The processor 302 is a hardware device for executing software instructions. The processor 302 may be any custom made or commercially available processor, a central processing unit (CPU), an auxiliary processor among several processors associated with the server 300, a semiconductor-based microprocessor (in the form of a microchip or chip set), or generally any device for executing software instructions. When the computer server 300 is in operation, the processor 302 is configured to execute software stored within the memory 310, to communicate data to and from the memory 310 including, for example, information regarding the character strengths, and to generally control operations of such server pursuant to the software instructions.

The I/O interfaces 304 may be used to receive user input from and/or for providing system output to one or more devices or components. User input may be provided via, for example, a keyboard, touch pad, and/or a mouse. System output may be provided via a display device and a printer (not shown). I/O interfaces 304 may include, for example, a serial port, a parallel port, a small computer system interface (SCSI), a serial ATA (SATA), iSCSI, a PCI Express interface (PCI-x), an infrared (IR) interface, a radio frequency (RF) interface, and/or a universal serial bus (USB) interface.

The network interface 306 may be used to enable the computer server 300 to communicate on a network, such as the Internet, the data network 105, the enterprise, and the like, etc. The network interface 306 may include, for example, an Ethernet card or adapter (e.g., 10BaseT, Fast Ethernet, Gigabit Ethernet, 10GbE) or a wireless local area network (WLAN) card or adapter (e.g., 802.11a/b/g/n). The network interface 306 may include address, control, and/or data connections to enable appropriate communications on the network. A data store 308 may be used to store data. The data store 308 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 308 may incorporate electronic, magnetic, optical, and/or other types of storage media. In one example, the data store 308 may be located internal to the server 300 such as, for example, an internal hard drive connected to the local interface 312 in the server 300. Additionally in another embodiment, the data store 308 may be located external to the computer server 300 such as, for example, an external hard drive connected to the I/O interfaces 304 (e.g., SCSI or USB connection). In a further embodiment, the data store 308 may be connected to the server 300 through a network, such as, for example, a network attached file server.

The memory 310 may include any of volatile memory elements (e.g., random access memory (RAM, such as DRAM, SRAM, SDRAM, etc.)), nonvolatile memory elements (e.g., ROM, hard drive, tape, CDROM, etc.), and combinations thereof. Moreover, the memory 310 may incorporate electronic, magnetic, optical, and/or other types of storage media. Note that the memory 310 may have a distributed architecture, where various components are situated remotely from one another, but can be accessed by the processor 302. The software in memory 310 may include one or more software programs, each of which includes an ordered listing of executable instructions for implementing logical functions. The software in the memory 310 may include a suitable operating system (O/S) 314 and one or more programs 320.

The operating system 314 essentially controls the execution of other computer programs, such as the one or more programs 320, and provides scheduling, input-output control, file and data management, memory management, and communication control and related services. The operating system 314 may be, for example, a version of Windows or Windows Server (both available from Microsoft, Corp. of Redmond, WA), LINUX (or another UNIX variant) (available from Red Hat of Raleigh, NC and various other vendors), Android and variants thereof (available from Google, Inc. of Mountain View, CA), Apple OS and iOS as well as respective variants thereof (available from Apple, Inc. of Cupertino, CA), or the like. The one or more programs 320 may be configured to implement the various processes, algorithms, methods, techniques, etc. described herein.

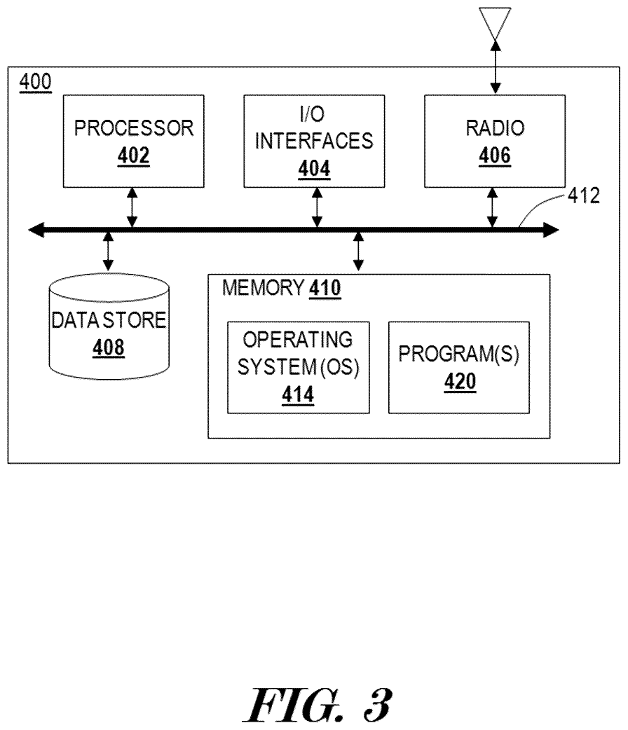

Referring to FIG. 3, an exemplary block diagram of a representative user or computing device 400 is depicted. The computing device 400 can be a digital device that, in terms of hardware architecture, generally includes a processor 402, input/output (I/O) interfaces 404, a radio 406, a data store 408, and memory 410. It should be appreciated by those of ordinary skill in the art that FIG. 3 depicts the computing device 400 in an oversimplified manner, and a practical embodiment may include additional components and suitably configured processing logic to support known or conventional operating features that are not described in detail herein. The components (402, 404, 406, 408, and 410) are communicatively coupled via a local interface 412. The local interface 412 can be, for example but not limited to, one or more buses or other wired or wireless connections, as is known in the art. The local interface 412 can have additional elements, which are omitted for simplicity, such as controllers, buffers (caches), drivers, repeaters, and receivers, among many others, to enable communications. Further, the local interface 412 may include address, control, and/or data connections to enable appropriate communications among the aforementioned components.

The processor 402 is a hardware device for executing software instructions. The processor 402 can be any custom made or commercially available processor, a central processing unit (CPU), an auxiliary processor among several processors associated with the computing device 400, a semiconductor-based microprocessor (in the form of a microchip or chip set), or generally any device for executing software instructions. When the computing device 400 is in operation, the processor 402 is configured to execute software stored within the memory 410, to communicate data to and from the memory 410, and to generally control operations of the computing device 400 pursuant to the software instructions. In an exemplary embodiment, the processor 402 may include a mobile optimized processor such as optimized for power consumption and mobile applications.

The I/O interfaces 404 can be used to receive data and user input and/or for providing system output. User input can be provided via a plurality of I/O interfaces 404, such as a keypad, a touch screen, a camera, a microphone, a scroll ball, a scroll bar, buttons, bar code scanner, voice recognition, eye gesture, and the like. System output can be provided via a display device such as a liquid crystal display (LCD), touch screen, and the like. The I/O interfaces 404 can also include, for example, a global positioning service (GPS) radio, a serial port, a parallel port, a small computer system interface (SCSI), an infrared (IR) interface, a radio frequency (RF) interface, a universal serial bus (USB) interface, and the like. The I/O interfaces 404 can include a graphical user interface (GUI) that enables a user to interact with the computing device 400. Additionally, the I/O interfaces 404 may be used to output notifications to a user and can include a speaker or other sound emitting device configured to emit audio notifications, a vibrational device configured to vibrate, shake, or produce any other series of rapid and repeated movements to produce haptic notifications, and/or a light emitting diode (LED) or other light emitting element which may be configured to illuminate to provide a visual notification.

The radio 406 enables wireless communication to an external access device or network. Any number of suitable wireless data communication protocols, techniques, or methodologies can be supported by the radio 406, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication. The data store 408 may be used to store data. The data store 408 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 408 may incorporate electronic, magnetic, optical, and/or other types of storage media.

The memory 410 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 410 may incorporate electronic, magnetic, optical, and/or other types of storage media. Note that the memory 410 may have a distributed architecture, where various components are situated remotely from one another, but can be accessed by the processor 402. The software in memory 410 can include one or more software programs, each of which includes an ordered listing of executable instructions for implementing logical functions. In the example of FIG. 3, the software in the memory system 410 includes a suitable operating system (O/S) 414 and programs 420.

The operating system 414 essentially controls the execution of other computer programs, and provides scheduling, input-output control, file and data management, memory management, and communication control and related services. The operating system 414 may be, for example, LINUX (or another UNIX variant), Android (available from Google), Microsoft Windows, iOS and Mac OS (available from Apple, Inc.), and the like. The programs 420 may include various applications, add-ons, etc. configured to provide end user functionality with the computing device 400. For example, exemplary programs 420 may include, but not limited to, a web browser, social networking applications, streaming media applications, games, mapping and location applications, electronic mail applications, financial applications, and the like. In a typical example, the trainee 101 typically uses one or more of the programs 420 along with a network 105 to manipulate information of the system 100.

The processing unit 402 may also include a main memory, such as a random access memory (RAM) or other dynamic storage device (e.g., dynamic RAM (DRAM), static RAM (SRAM), and synchronous DRAM (SDRAM)), coupled to the bus for storing information and instructions to be executed by the processor 402. In addition, the main memory 410 may be used for storing temporary variables or other intermediate information during the execution of instructions by the processor 402. The processor 402 may further include a read only memory (ROM) or other static storage device (e.g., programmable ROM (PROM), erasable PROM (EPROM), and electrically erasable PROM (EEPROM)) coupled to the bus for storing static information and instructions for the processor 402.

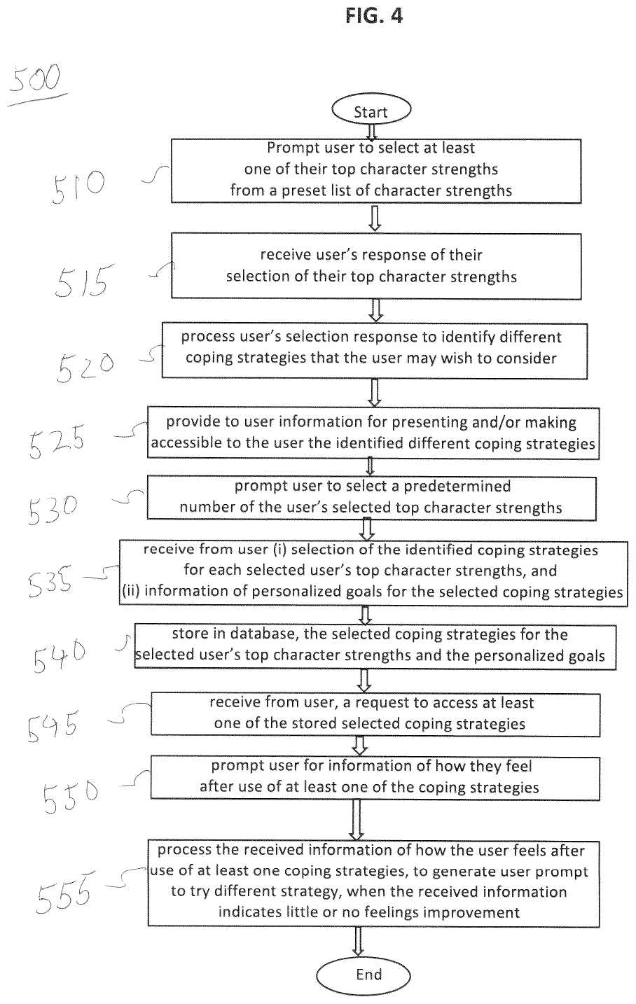

FIG. 4 depicts an exemplary flow diagram of a representative method 500 for strength-based coping training of a trainee, i.e., user, in accordance with the present invention. Such method is particularly advantageous for those individuals on the autism spectrum. The method 500 starts with step 510 by execution of a software application on the computing device 400 depicted in FIG. 3, to prompt the trainee (also referred to herein as the “user”) to select at least one, but preferably three to five, of their top character strengths from a preset list of character strengths. This preset list of character strengths being identified in a character strengths database accessible by the computing device 400 via execution of the software application. In step 515, the computing device 400 receives the user's corresponding selection of at least one of their top character strengths from the preset list of character strengths.

Then, in step 520, the computing device 400 processes the received selected top character strengths of the user to identify different coping strategies that the user may wish to consider from a coping strategy database associated with the software application, wherein the identified different coping strategies are based on the received user's selection of their top character strengths. Example coping strategies that the user may wish to consider may include: 1. “Educate yourself on the problem you are having before making a decision,”; 2. “Ask someone else to tell you about their point of view; ” 3. “Remember a time that you persevered in the past and how good it felt; ” and/or 4. “Do something challenging even when scared.”

The method 500 then proceeds to step 525 , where the software application provides the computing device information for at least one of presenting or making access to the user the identified different coping strategies that the user may wish to consider, and in step 530 prompts user to select a predetermined number of the identified coping strategies for at least one of the selected user's top character strengths.

In step 535, the computing device 400 receives from the user (i) the user's selection of the identified coping strategies for each of their selected user's top character strengths, and (ii) information indicative of personalized goals with regard to use of the selected coping strategies. In step 540, the software application causes the computing device 400 to store in a user database the user's selected coping strategies for each of the selected user's top character strengths and the information indicative of personalized goals with regard to use of the selected user's top character strengths.

Then, in step 545, the computing device 400 receives from the user, a request to access at least one of the stored selected coping strategies. At some time thereafter, the software application causes the computing device 400, in step 550, to prompt the user to provide information of how the user feels after use of at least one of the coping strategies. Exemplary prompts may include “How do you feel now? ” and if the response is that the user still feels bad, then a subsequent prompt may be, for example, “that's okay, maybe you can try another strength, or another strategy, you can also try again, one more time.”. Lastly, in step 555, the software application may cause the computing device 400 to process the received information of how the user feels, and thereafter prompts the user to try at least one different coping strategy, when the received information of how the user feels is indicative of relatively little or no improvement in feelings.

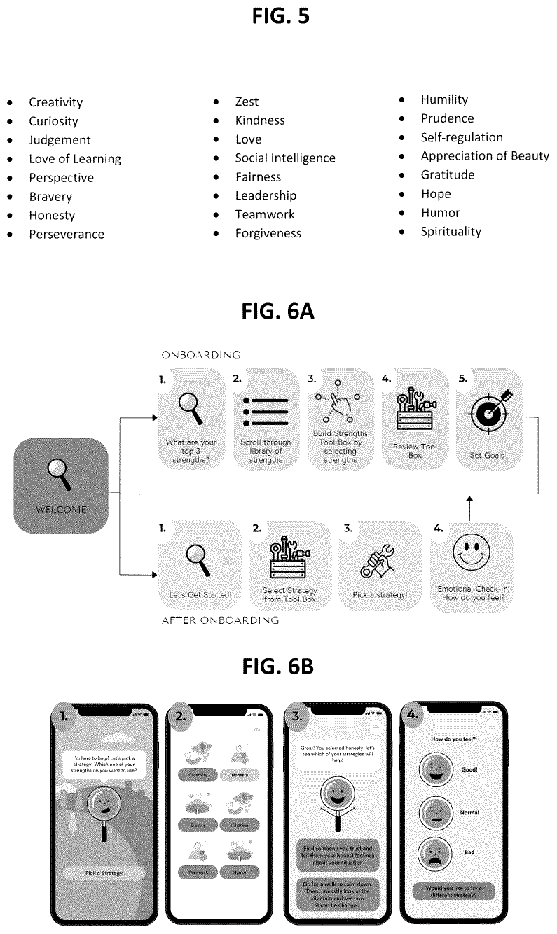

The software application performing the steps 510-515 may be alternatively performed by the computer server 300 of FIG. 1, or a different computer server operated by the operator of the computer server 300 or a different operator such as, for example, t he VIA Institute on Character that offers the VIA Inventory of Strengths (“VIA-IS”) is for people aged 18 and older, while the VIA Inventory of Strengths-Youth Version is a self-report questionnaire intended to measure how one expresses character strengths, and is accessible at www.VIAcharacter.org. The VIA Institute on Character provides such services for 24 different character strengths, represented by the chart in FIG. 5. However, the software application in accordance with the present invention may employ a larger or smaller number of character strengths. Lastly, the software application may be a fully-or partially-contained software program or programs transmitted to and executed by the computing device 400.

Although the method 500 depicts an exemplary embodiment of the present invention with the execution of a software applications performing steps 510-555, it should be understood that such steps may be alternatively performed by multiple software applications executed on the computing device 400 or a larger number of network-accessible computer servers.

The method 500 may additionally perform the step (not shown) of generating a monthly summary of which strengths were employed by user within a previous particular time period, such as for example, the past month or quarter. Such summary report may be generated based on the strengths employed by user as maintained in the data store 308, and may be sent or made accessible to the user automatically at the end of a month, quarter or other designated or requested time period.

FIG. 6A depicts a diagram of an exemplary implementation sequence for onboarding users and after onboarding, execution of a representative version of the method 500. FIG. 6B depicts an exemplary sequence display screens useable on a mobile user device for performing the method 500.

In accordance with another embodiment of the method 400 of FIG. 4, an optional step (not shown in FIG. 4) causes a notice message to be transmitted intermittently for display on the computing device 400 as reminder for the user 101 to update a strength-spotting journal with entries identifying at least one character strength that the user employed over a previous particular time period such as, for example, earlier that day, the prior day or within the past few days or week. The entry would further entered by the user 101 would further include how the user employed such at least one character strength over the previous time period.

Many different methods may be employed to facilitate entering such information into the strength-spotting journal including, for example, of enabling the user to launch a journal application via the computing device 400 of the user 101.

In accordance with another embodiment of the method 400 of FIG. 4, an optional step (not shown in FIG. 4) causes the execution, via the computing device 400 of the user 101, a toolbox application wherein at least one of the toolbox application or the user 101 identifies at least one of the coping strategies to be added to a database associated with the toolbox application (the “toolbox database”), wherein the user would identify at least one task employing the at least one of the coping strategies added to the toolbox database. Such toolbox database may be a portion of the data store 308 of FIGS. 1 and 2. A summary and/or description of such identified at least one task may be entered by the user 101 into the toolbox database or some other database. It would be advantageous for the entered at least one task to arise from or be associated with situations encountered for at least one of school, work place, family, and friends. The task may also be for fun of the user 101, which could further entice the user 101 to more frequently interact with the executed toolbox application and/or engage such application with greater focus and attention.

A representative operation of an exemplary toolbox application would include the user 101, when upset, accessing the toolbox application via an optional step of the method 400 or the computer or mobile device application, such as an app. The user 101 may then access the coping strategies they previously entered, and select one of the strategies to employ in an attempt to overcome or mitigate being upset. The user 101 would then perform an exercise in accordance with the selected coping strategy. The method 400, app and/or toolbox application would then generate a prompt to the user 101 inquiring whether they feel better. If the user 101 enters a response of “yes,’ the prompting would end until the next time the method 400, app and/or toolbox application is executed. However, if the user 101 enters a response of “no,’ the method 400, app and/or toolbox application would then prompt the user 101 to try a different coping strategy in the toolbox database or some other means to feel better. This routine could be repeated several times until the user 101 finds a coping strategy that makes the user 101 feel better.

A specific example of an operation of an exemplary toolbox application would be when the user 101 is upset because she burned the toast she was preparing for breakfast. The user 101 would then execute the toolbox application by one of the means described above to access her stored coping strategies. Because she previously entered coping strategies based on the character strength “love”, such as “Call someone you love and tell them how you are feeling,” she can select that coping strategy and based on such selection, she calls her husband and tell him about how upset she is because of the burnt toast. He sympathizes with her feeling, but it did make her feel better. She responds to the apps inquiry as to how she is feeling by responding she is still upset. The app then suggests she try a different coping strategy, and she selects another coping strategy based on her character strength of “love,” such as “think about someone you love. ” In accordance with that strategy, she thinks about her mother and how much she loves her, and begins to feel better. Then, when prompted by the app regarding how she is feeling, she responds “yes,” and the prompting would end, and she goes on with her day without being upset about, or the distraction of focusing on, the toast she burned.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a propagated signal or a computer readable medium. The propagated signal is an artificially generated signal, e.g., a machine generated electrical, optical, or electromagnetic signal that is generated to encode information for transmission to suitable receiver apparatus for execution by a computer. The computer readable medium can be a machine readable storage device, a machine readable storage substrate, a memory device, a composition of matter effecting a machine readable propagated signal, or a combination of one or more of them.

A computer program (also known as a program, software, software application, application, script, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network. It should be readily understood that although the embodiments have been described with respect to FIGS. 1-4, as a server-based implementation with the user device 400 using an software application, i.e., an App, or browser application, it as alternatively possible for the certain ones or all of the operations performed by the server, to be performed by the user device 400 by execution of corresponding software code stored in, or accessible by the user device 400.

Additionally, the logic flows and structure block diagrams described in this patent document, which describe particular methods and/or corresponding acts in support of steps and corresponding functions in support of disclosed structural means, may also be utilized to implement corresponding software structures and algorithms, and equivalents thereof. The processes and logic flows described in this specification can be performed by one or more programmable processors (computing device processors) executing one or more computer applications or programs to perform functions by operating on input data and generating output.

Processors suitable for the execution of a computer program include, by way of example, both general and special purpose microprocessors, and any one or more processors of any kind of digital computer.

Computer readable media suitable for storing computer program instructions and data include all forms of non-volatile memory, media and memory devices, including by way of example semiconductor memory devices, e.g., EPROM, EEPROM, flash memory devices, and solid state drives; magnetic disks, e.g., internal hard disks or removable disks; and CD ROM and DVD ROM disks. The processor and the memory can be supplemented by, or incorporated in, special purpose logic circuitry.

To provide for interaction with a user, embodiments of the subject matter described in this specification can be implemented on a computer having a display device, e.g., a LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the subject matter described in this specification can be implemented in a computer server that includes a back end component, e.g., as a data server, or that includes a middleware component, e.g., an application server, or that includes a front end component, e.g., a client computer having a graphical user interface or a Web browser through which a user can interact with an implementation of the subject matter described is this specification, or any combination of one or more such back end, middleware, or front end components. The components of the server can be interconnected by any form or medium of digital data communication, e.g., a communication network. Examples of communication networks include a local area network (“LAN”) and a wide area network (“WAN”), e.g., the Internet.

The computer server can include clients and servers. A client and server are generally remote from each other and typically interact through a communication network or the cloud. The relationship of client and server arises by virtue of computer programs running on the respective computers and having a client server relationship to each other.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The computer server 300 may perform a portion or all of the processing steps of the invention in response to the processor executing one or more sequences of one or more instructions contained in a memory, such as the main memory. Such instructions may be read into the main memory from another computer readable medium, such as a hard disk or a removable media drive. One or more processors in a multi-processing arrangement may also be employed to execute the sequences of instructions contained in main memory.

Stored on any one or on a combination of computer readable media, the present invention includes execution of software for controlling the computer server, alone or in combination with other devices for implementing the invention, and for enabling the computer system to interact with a human user. Such software may include, but is not limited to, device drivers, operating systems, development tools, and applications software. Such computer readable media further includes the computer program product of the present invention for performing all or a portion (if processing is distributed) of the processing implementing in the invention.

The software code of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

The invention is further described by the following numbered paragraphs:

1. A method for strength-based coping training, the method comprising the steps of:
a. executing a software application operable on a computing device to prompt a user to select at least one of their top character strengths from a preset list of character strengths, said preset list of character strengths being identified in a character strengths database accessible by the software application;
b. the computing device receiving from the user in response to the prompting by the software application, a selection of at least one of their top character strengths from the preset list of character strengths;
c. the computing device processing the received selection of the user's top character strengths to identify different coping strategies that the user may wish to consider from a coping strategy database associated with the software application, said identification of the different coping strategies being based on the received selection of the user's top character strengths;
d. obtaining by the computing device information for at least one of presenting or providing access to the user at least one of the identified different coping strategies that the user may wish to consider;
e. providing a prompt to the user to select a predetermined number of the identified coping strategies for at least one of the selected user's top character strengths;
f. the computer device receiving from the user (i) the user's selection of the identified coping strategies for each of the selected user's top character strengths, and (ii) information indicative of personalized goals with regard to use of the selected coping strategies;
g. the computer device storing in a user database the user's selected coping strategies for each of the selected user's top character strengths and the information indicative of personalized goals with regard to use of the selected user's top character strengths;
h. the computing device receiving from the user, a request to access at least one of the stored selected coping strategies;
i. prompting the user to provide information of how the user feels after use of at least one of the coping strategies;
j. receiving from the user information of how the user feels after use of at least one of the coping strategies;
k. processing, by the computing device, the received information of how the user feels after use of at least one of the coping strategies, and
l. prompting the user to try different one of the at least one of the coping strategies, when the received information in step j. is indicative of little or no improvement in the user's feelings.

2. The method of paragraph 1, wherein the preset list of character strengths includes twenty-four character strengths.

3. The method of paragraph 1, wherein the prompting by a software application in step
a. further comprises prompting the user to identify five of their top character strengths.

4. The method of paragraph 1, further comprising the step of generating a of which strengths were used in a previous particular time period.

5. The method of paragraph 4, where the previous particular time period is one of weekly, monthly or quarterly.

6. The method of paragraph 1, wherein the method is specifically adapted for coping training of users on the autism spectrum.

7. The method of paragraph 1, further comprising the step of causing a notice to be transmitted intermittently to update a strength-spotting journal with entries identifying at least one character strength that the user employed over a previous particular time period and how the user employed such at least one character strength.

8. The method of paragraph 7, further comprising the steps of enabling the user to launch a journal application via the computing device of the user.

9. The method of paragraph 7, wherein the previous particular time period is at least one of that day or the previous day.

10. The method of paragraph 1, further comprising the steps of executing via the computing device of the user, a toolbox application wherein at least one of the toolbox application or the user identifies at least one of the coping strategies to be added to a database associated with the toolbox application, wherein the user would identify at least one task employing the at least one of the coping strategies added to the toolbox database.

11. The method of paragraph 10, wherein the at least one task is associated with situations encountered for at least one of school, work place, family and for fun.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A computer-implemented method for strength-based coping training, the method comprising the steps of:
a. executing a software application operable on a computing device to prompt a user to select at least one of their top character strengths from a preset list of character strengths, said preset list of character strengths being identified in a character strengths database accessible by the software application;
b. the computing device receiving from the user in response to the prompting by the software application, a selection of at least one of their top character strengths from the preset list of character strengths;
c. the computing device processing the received selection of the user's top character strengths to identify different coping strategies that the user may wish to consider from a coping strategy database associated with the software application, said identification of the different coping strategies being based on the received selection of the user's top character strengths;
d. obtaining by the computing device information for at least one of presenting or providing access to the user at least one of the identified different coping strategies that the user may wish to consider;
e. providing a prompt to the user to select a predetermined number of the identified coping strategies for at least one of the selected user's top character strengths;
f. the computer device receiving from the user (i) the user's selection of the identified coping strategies for each of the selected user's top character strengths, and (ii) information indicative of personalized goals with regard to use of the selected coping strategies;
g. the computer device storing in a user database the user's selected coping strategies for each of the selected user's top character strengths and the information indicative of personalized goals with regard to use of the selected user's top character strengths;
h. the computing device receiving from the user, a request to access at least one of the stored selected coping strategies;
i. prompting the user to provide information of how the user feels after use of at least one of the coping strategies;
j. receiving from the user information of how the user feels after use of at least one of the coping strategies;
k. processing, by the computing device, the received information of how the user feels after use of at least one of the coping strategies, and
l. prompting the user to try different one of the at least one of the coping strategies, when the received information in step j. is indicative of little or no improvement in the user's feelings.

2. The method of claim 1, wherein the preset list of character strengths includes twenty-four character strengths.

3. The method of claim 1, wherein the prompting by the computing device in step a. further comprises prompting the user to identify three to five of their top character strengths.

4. The method of claim 1, further comprising the step of generating a list of which character strengths were used in a previous particular time period.

5. The method of claim 4, wherein the previous particular time period is one of weekly, monthly or quarterly.

6. The method of claim 1, wherein the method is specifically adapted for coping training of users on the autism spectrum.

7. The method of claim 1, further comprising the step the computing device intermittently providing alerts to the user to update a strength-spotting journal with entries identifying at least one character strength that the user employed over a previous particular time period and how the user employed such at least one character strength.

8. The method of claim 7, further comprising the steps of enabling the user to launch execution of a journal application via the computing device.

9. The method of claim 7, wherein the previous particular time period is at least one of that day, the previous day or days, and/or previous week.

10. The method of claim 1, further comprising the step of the computing device executing a toolbox application wherein at least one of the toolbox application or the user identifies at least one of the coping strategies to be added to a database associated with the toolbox application, wherein the user would identify at least one task employing the at least one of the coping strategies added to the toolbox database.

11. The method of claim 10, wherein the at least one task is associated with situations encountered for at least one of school, work place, family and for fun.
